(19) United States
(12) Patent Application Publication
(10) Pub. No.: US 2021/0315513 A1
(43) Pub. Date: Oct. 14, 2021

(54) DENTAL IMAGING SYSTEM

(71) Applicant: Ivoclar Vivadent AG, Schaan (LI)

(72) Inventors: Stéphane Follonier, Vilters (CH); Wolfgang Wachter, Schaan (LI)

(21) Appl. No.: 17/288,603

(22) PCT Filed: Oct. 18, 2019
(86) PCT No.: PCT/EP2019/078375

§ 371 Date: Apr. 26, 2021

(30) Foreign Application Priority Data
Nov. 12, 2018 (EP) 18205618.4

(51) Int. Cl. A61B 5/00

(52) U.S. Cl. CPC A61B 5/4547; A61B 5/742; A61B 5/0066; A61B 5/0071

(57) ABSTRACT

The present invention relates to a dental imaging system (100) having an electronic camera (103) for capturing a digital image of a luminescence region (111); and data glasses (105) for displaying the captured image on top of a visually perceived image.

The present invention relates to a dental imaging system and to a method for dental imaging.

The visual capturing of a tooth situation inside an oral cavity under luminescence can be difficult, since luminescence regions can often be recognized only with difficulty in daylight.

It is the technical object of the present invention to provide improved image data for a dental diagnosis.

This object is achieved by the subject matter of the independent claims. Advantageous embodiments are subject of the dependent claims, the description and the figures.

According to a first aspect this object is achieved by a dental imaging system, having an electronic camera for capturing a digital image of a luminescence region; and data glasses for displaying the captured image on top of a visually perceived image. The imaging system can comprise a light source for exciting luminescence inside an oral cavity. The excitation of the luminescence of tooth substance can be carried out with or without additional dye. By means of the dental imaging system the technical advantage is achieved so that the luminescence region can be optically perceived more satisfactorily.

In a technically advantageous embodiment of the imaging system, the dental imaging system comprises a calculation device which is designed to increase a contrast or an intensity of the captured luminescence region in the digital image. In this way for example the technical advantage is achieved that luminescence regions can be optically perceived still more clearly. By visual superimposition on the illuminated tooth by means of the data glasses the luminescence region can be illustrated in real time and recognized directly during a medical examination.

In a further technically advantageous embodiment of the imaging system the dental imaging system comprises a calculation device which is designed to integrate depth information into the digital image in order to increase the basis of the digital image. In this way, for example also the technical advantage is achieved that luminescence regions can be optically perceived still more clearly or that a depth of the carious lesion can be indicated.

In a further technically advantageous embodiment of the imaging system, a light source for exciting luminescence and/or the electronic camera is integrated in the data glasses or in a dental treatment device. In this way, for example the technical advantage is achieved that the light source can be held at suitable positions.

In a further technically advantageous embodiment of the imaging system, the light source is designed to emit monochromatic, non-monochromatic, non-polarised or polarised light. In this way, for example the technical advantage is achieved that suitable light is used to capture the luminescence. The light can be emitted continuously or emitted in a pulse manner.

In a further technically advantageous embodiment of the light source, the monochrome filter, the bandpass filter or the polarisation filter can be moved as required into the beam path of the electronic camera. The monochrome filter, the bandpass filter or the polarisation filter can also be controlled electronically.

In a further technically advantageous embodiment of the imaging system the light has a wavelength in the range of 300 to 480 nm. However, the light can also have a wavelength in the range of 330 to 400 nm. The light can have for example a wavelength of 244 nm, 257 nm, 325 nm, 364 nm in the ultraviolet range, a wavelength of 457 nm, 473 nm, 488 nm, 514 nm, 532 nm, 633 nm, 660 nm in the visible range or a wavelength of 785 nm, 830 nm, 980 nm, 1064 nm in the infrared range. In this way for example the technical advantage is achieved that the luminescence of tooth substance can be excited in a particularly satisfactory manner.

In a further technically advantageous embodiment of the imaging system, the electronic camera comprises a monochrome filter, a bandpass filter or a polarisation filter. In this way, for example the technical advantage is achieved that the imaging by the camera can be improved.

In a further technically advantageous embodiment of the imaging system, the monochrome filter, the bandpass filter or the polarisation filter can be moved as required into the beam path of the electronic camera. In this way, for example the technical advantage is achieved that images can be obtained with and without these filters and can then be compared with each other.

The monochrome filter, the bandpass filter or the polarisation filter can also be controlled electronically. Electronic control of these filters can be achieved for example using a voltage by which the optical properties of these filters can be adjusted or influenced in a targeted manner. For example, in this way, a wavelength, a wavelength range or a polarisation angle of the filters can be controlled in a targeted manner.

This electronic control permits for example self-optimisation of the contrast or the intensity in order to improve the information content of the digital image. For this purpose, an electronic control device can be provided which carries out this control.

In a further technically advantageous embodiment of the imaging system, the imaging system comprises a light source for exciting the luminescence, which is designed to emit collimated light. In this way, for example the technical advantage is achieved that the light beams can be directed to and concentrated on a specific region.

In a further technically advantageous embodiment of the imaging system, the collimation angle is between 0°-2° or 0°-4°. In this way, for example also the technical advantage is achieved that the light beams can be directed to and concentrated on a specific region.

In a further technically advantageous embodiment of the imaging system, the imaging system comprises a light source for exciting the luminescence, which is formed by a light-emitting diode, a laser diode or a laser. In this way, for example also the technical advantage is achieved that particularly suitable light sources are used.

According to a second aspect this object is achieved by a method for dental imaging, comprising the steps of capturing a digital image of a luminescence region by means of an electronic camera; and displaying the captured image on top of a visually perceived image on data glasses. In this way, the same technical advantages are provided as by the imaging system according to the first aspect.

In a technically advantageous embodiment of the method for dental imaging, a contrast or an intensity of the captured luminescence region in the digital image is increased. In this way, for example also the technical advantage is achieved that luminescence regions can be optically perceived still more clearly.

In a further technically advantageous embodiment of the method, depth information is integrated into the digital image in order to increase the basis of the digital image. In this way, for example also the technical advantage is achieved that luminescence ranges can be optically perceived still more clearly.

In a further technically advantageous embodiment of the method for dental imaging, the light source emits monochromatic, non-monochromatic, non-polarised or polarised light. In this way, for example also the technical advantage is achieved that suitable light is used to capture the luminescence.

In a further technically advantageous embodiment of the method for dental imaging, the light source emits collimated light. In this way for example also the technical advantage is achieved that the light beams can be directed to and concentrated on a specific region.

In a further technically advantageous embodiment of the method, an emitted wavelength, an electronic filter and/or the electronic camera are controlled in such a way that an automatic increase or optimisation of the contrast of the digital image or an increase in the sensitivity of the camera takes place. In this way, for example self-optimisation of the contrast or of the intensity of the luminescence region can be effectuated. In this way for example also the technical advantage is achieved that luminescence regions can be perceived more quickly and clearly. This self-optimisation can be carried out by suitable algorithms which control the emitted wavelength, the electronic filter and/or the electronic camera based on the captured image.

Exemplified embodiments of the invention are illustrated in the drawings and are described in more detail hereinunder.

In the figures:

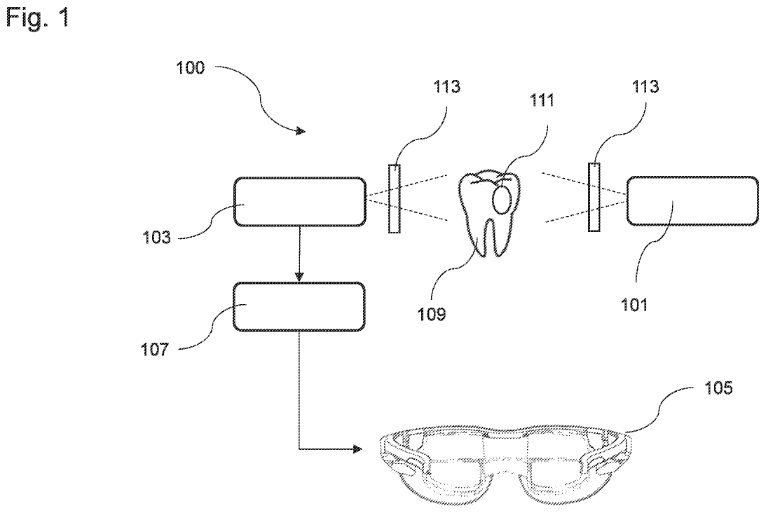

FIG. 1 shows a schematic view of a dental imaging system; and

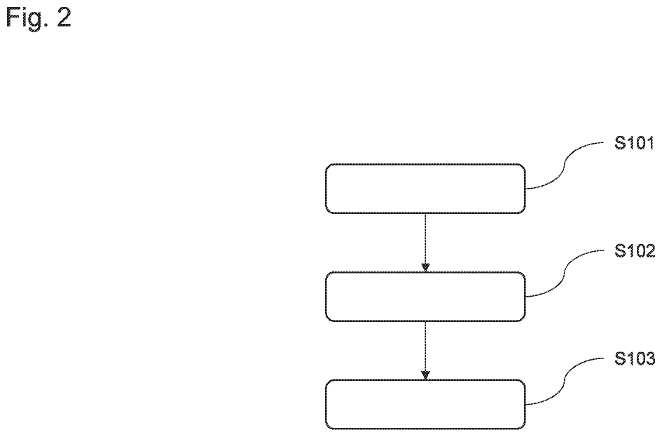

FIG. 2 shows a block diagram of a method for dental imaging.

FIG. 1 shows a schematic view of a dental imaging system 100. The imaging system 100 serves to obtain images of the oral cavity of a patient. The imaging system 100 optionally comprises a light source 101 for exciting a luminescence inside the oral cavity, an electronic camera 103 for capturing a digital image of a luminescence region 111; and data glasses 105 for displaying the captured image on top of a visually perceived image. By means of a digital processing device the luminescence region 111 can be optically accentuated or enhanced in the captured image so that this can be recognized better.

The luminescence comprises fluorescence, phosphorescence and a Raman effect. Fluorescence is the spontaneous emission of light shortly after the excitation of a material by electronic transitions. In this case the emitted light is generally lower in energy than the light previously absorbed by the light source 101. Phosphorescence describes a longer afterglow following the optical excitation. The Raman effect is the inelastic scattering of light on molecules.

The fluorescence, phosphorescence or Raman effect causes porosity, roughness or gloss values of the tooth substance to become visible. It can thus be established whether or not a tooth 109 is carious or in which other condition it is.

The light source 101 is formed for example by a light-emitting diode, a laser diode or a laser. The light source 101 can emit light in the wavelength range of 300 nm to 480 nm or 330 nm to 400 nm. The light beam of the light source can be collimated, i.e. orientated substantially parallel so that the light can be concentrated on a specific region inside the oral cavity. This can be achieved for example in a simple manner by a collimator or collecting lens in front of the light source or a laser as a light source 101.

The light source 101 can be formed as a separate apparatus which is provided only for the purpose of illumination or can be integrated in another dental treatment tool, such as a scanner, a probe or a mirror. The light source 101 can emit monochromatic, non-monochromatic, non-polarised or polarised light. In general, the light source 101 can emit any form of light which excites luminescence.

The luminescence region 111 (ROI—region of interest) is recorded by an electronic camera 103 with a CMOS chip, which captures a digital image of the luminescence and generates corresponding image data. The digital image and the image data can be processed by information technology so that the intended luminescence effect is additionally highlighted. The processing of the digital image can be carried out by an image analysis which permits a distinction—acquired by machine learning—between luminescence zones within the luminescence region 111 which are visually barely distinguishable.

In addition, a monochrome filter 113, a bandpass filter 113 or a polarisation filter 113 can be moved or folded into the beam path of the camera 103 for example upstream of the camera 103 and/or upstream of the light source 101. A monochrome filter allows passage only of light of a specific wavelength. A bandpass filter is a filter which allows passage only of light of a specific frequency range. A polarisation filter allows passage only of light of a specific polarisation direction.

In this way, images can be obtained with and without these filters 113 and can then be compared with each other. The obtained images can be subjected to a difference analysis.

The electronic camera 103 can capture for example wavelengths in the short-wave range of light below 400 nm or in the longer wavelength range over 700 nm which cannot be perceived by the eye. In this way, non-visible fluorescence, phosphorescence or Raman effects can also be captured.

A calculation device 107 of the dental imaging system 100 can carry out processing of the image data and increase the contrast or the intensity of the captured luminescence region in the digital image. For this purpose, luminescence regions 111 in the digital image can be identified and processed by means of graphics algorithms. It is thus possible to enhance weak fluorescence effects, phosphorescence effects or Raman effects or to provide them with other colour values which can be perceived better. Identification of the luminescence region 111 can be effected by detecting characteristic wavelengths/colours of the luminescence in the recorded image.

The calculation device 107 is formed for example by a software module which is executed by a processor. The original and processed image data can be stored in a digital memory.

In addition, the calculation device 107 can integrate depth information into the digital image in order to increase the basis of the digital image. The depth information can be depicted by grey scales and a spatial or three-dimensional condition of the tooth can be depicted. The depth information can be for example an image obtained by optical coherence tomography (OCT) or a radiographic method. This depth information can additionally be projected onto the data glasses 105. The captured luminescence regions can be superimposed on the depth information. In this way, additional information can be provided which leads to an improved diagnosis.

The image or the image data can then be indicated to the user on the data glasses 105 so that luminescence regions 111 can be perceived more satisfactorily. The data glasses 105 obtain the image data of the recorded image and are an electronic apparatus worn as glasses by which fluorescence effects, phosphorescence effects or Raman effects can be optically displayed to a user in addition to the natural visual perception of the tooth region.

By means of the display device, the data glasses 105 can superimpose additional information on an image which is perceived by the wearer's eye. Special software algorithms can be used for visual superimposition on the visually perceived image. The display device of the data glasses 105 comprises for example a display screen close to the eyes or a projector for directly projecting on the retina. In a corresponding manner, this can also be performed on a wearable apparatus.

In addition, a zoom function can be integrated into the imaging system 100 in order to be able to enlarge the captured image over the visually perceived image and thus render the luminescence regions 111 more visible. The imaging system 100 can be voice-controlled or motion-controlled by means of the data glasses 105 so that self-optimisation of the intensity or the contrast of the captured image can be carried out.

FIG. 2 shows a block diagram of a method for dental imaging. The method includes step S101 of exciting the luminescence inside an oral cavity by means of the light source 101, step S102 of capturing the digital image of the luminescence by means of an electronic camera 103; and step S103 of displaying the captured image on top of a visually perceived image on data glasses 105. In this way, luminescence effects can be rendered more clearly visible during observation of the luminescence region 111.

All features explained and illustrated in conjunction with individual embodiments of the invention can be provided in different combinations in the subject-matter according to the invention in order to achieve their advantageous effects simultaneously.

All the method steps can be implemented by devices which are suitable for carrying out the respective method step. All functions which are carried out by apparatus features can be a method step of a method.

The scope of protection of the present invention is defined by the claims and is not limited by the features explained in the description or shown in the figures.

### LIST OF REFERENCE SIGNS

100 dental imaging system
101 light source
103 electronic camera
105 data glasses
107 calculating device
109 tooth
111 luminescence region
113 monochrome filter/bandpass filter/polarisation filter

1. A dental imaging system (100), having:
an electronic camera (103) for capturing a digital image of a luminescence region (111); and
data glasses (105) for displaying the captured image on top of a visually perceived image.

2. The imaging system (100) as claimed in claim 1,
wherein the dental imaging system (100) comprises a calculation device (107) which is designed to increase a contrast or an intensity of the captured luminescence region (111) in the digital image.

3. The imaging system (100) as claimed in claim 1,
wherein the dental imaging system (100) comprises a calculation device (107) which is designed to integrate depth information into the digital image in order to increase the basis of the digital image.

4. The imaging system (100) as claimed in claim 1,
wherein a light source (101) for exciting luminescence and/or the electronic camera (103) is integrated in the data glasses (105) or in a dental treatment device (109).

5. The imaging ag system (100) as claimed in claim 1,
wherein the light source (101) is designed to emit monochromatic, non-monochromatic, non-polarised or polarised light.

6. The imaging system (100) as claimed in claim 5,
wherein the light has a wavelength in the range of 300 to 480 nm.

7. The imaging system (100) as claimed in claim 1,
wherein the electronic camera (103) and/or the light source (101) comprises a monochrome filter (113), a bandpass filter (113) or a polarisation filter (113).

8. The imaging system (100) as claimed in claim 7,
wherein the monochrome filter (113), the bandpass filter (113) or the polarisation filter (113) is configured to be controlled or to be moved as required into the beam path of the electronic camera (103) or of the light source (101).

9. The imaging system (100) as claimed in claim 1,
wherein the imaging system (100) includes a light source (101) for exciting luminescence, which is designed to emit collimated light.

10. The imaging system (100) as claimed in claim 9,
wherein the collimation angle is between 0°-2° or 0°-4°.

11. The imaging system (100) as claimed in claim 1,
wherein the imaging system (100) includes a light source (101) for exciting luminescence, which is formed by a light-emitting diode, a laser diode or a laser.

12. A method for dental imaging (100), comprising the steps of:
capturing (S102) a digital image of a luminescence region (111) by means of an electronic camera (103); and
displaying (S103) the captured image on top of a visually perceived image on data glasses (105).

13. The method as claimed in claim 12,
wherein a contrast or an intensity of the captured luminescence region (111) in the digital image is increased.

14. The method as claimed in claim 12,
wherein depth information is integrated into the digital image in order to increase the basis of the digital image.

15. The method as claimed in claim 12,
wherein an emitted wavelength, an electronic filter (113) and/or the electronic camera (103) are controlled in such a way that an automatic increase in the contrast of the digital image or an increase in the sensitivity of the camera (103) takes place.
